(19) United States
(12) Patent Application Publication
(10) Pub. No.: US 2008/0057125 A1
(43) Pub. Date: Mar. 6, 2008

(54) STABLE TABLET DOSAGE FORMS OF PROTON PUMP INHIBITORS

(76) Inventors: Ranga R. Namburi, Plainsboro, NJ (US); Rama Prasad Karri, Plainsboro, NJ (US); Ravi Srikanth Tallapragada, Plainsboro, NJ (US); Buroise F. Palkhiwala, East Windsor, NJ (US)

Correspondence Address: Richard S. Roberts;Roberts & Roberts, L.L.P., Attorneys At Law, P.O. Box 484, Princeton, NJ, 08542-0484, US

(21) Appl. No.: 11/849,505

(22) Filed: Sep. 4, 2007

Related U.S. Application Data
(62) Division of application No. 11/082,610, filed on Mar. 17, 2005.

(51) Int. Cl. A61K 9/30; A61K 31/4439; A61K 9/36

(52) U.S. Cl. 424/475; 424/465; 424/480; 514/338

(57) ABSTRACT

This invention relates to a method of making oral formulations of practically water insoluble, or very slightly water soluble proton pump inhibitors, the oral dosage forms so made, and methods of use thereof. The oral dosage form has a core tablet of compressed particles composed of powder particles of a pharmaceutically acceptable material, having coated thereon admixture of an amorphous, salt form of a benzimidazole proton pump inhibitor produced in-situ; and a pharmaceutically acceptable, water-soluble, hydrophilic polymer having a surfactant functionality. The coated core tablet has a pharmaceutically acceptable sub-coating on the core tablet; and a pharmaceutically acceptable enteric coating on the sub-coating. The coated tablet may provide enhanced absorption when administered orally.

### BACKGROUND OF THE INVENTION

### Field of the Invention

The present invention concerns methods of making oral formulations of practically water insoluble, or very slightly water soluble proton pump inhibitors, the oral dosage forms so made, and methods of use thereof.

The proton pump, located in the apical membrane of the parietal cell, is responsible for the secretion of acid in the stomach when it is stimulated by the enzyme adenosine triphosphate (H⁺, K⁺)-ATPase. Proton pump inhibitors are a class of anti-secretory compounds used in the management of gastrointestinal disorders. They suppress gastric acid secretion by the specific inhibition of the (H⁺, K⁺)-ATPase enzyme system at the secretory surface of the gastric parietal cell.

A family of substituted benzimidazoles has been developed as specific proton pump inhibitors (PPIs). Thus, PPIs are well known in the art as gastric acid secretion inhibiting agents. Since the introduction of omeprazole (Prilosec™) in 1989, several other PPIs have become available that include Lansoprazole (Prevacid™), Rabeprazole (Aciphex™), Pantoprazole (Protonix™) and Esomeprazole (Nexium™). PPIs are inactivated by exposure to gastric juice and are delivered in delayed-release gelatin capsules containing enteric-coated granules (omeprazole and lansoprazole) or in delayed-release enteric-coated tablets (rabeprazole and patoprazole) or in delayed-release enteric-coated granules compressed in to tablet dosage forms (omeprazole, lansoprazole and esomeprazole). Also an intravenous form of pantoprazole is now available. U.S. Pat. No. 4,255,431 describes a compound 2-[2-(3,5-dimethyl-4-methoxy)-pyridyl methyl sulfinyl]-(5-methoxy)-benzimidazole (Omeprazole) or pharmaceutically acceptable salt or non-toxic acid addition salt as a therapeutic compound for mammals including man, suffering from gastric acid secretion disturbances. U.S. Pat. No. 4,628,098 discloses that Lansoprazole is a substituted benzimidazole 2-[[[3-methyl-4-(2,2,2-trifluoroethoxy)-2-pyridyl]methyl]sulfinyl]benzimidazole, a compound and a pharmacologically acceptable salt thereof that inhibits gastric acid secretion. Omeprazole is useful as well for providing gastrointestinal cytoprotective effects in mammals and man. Omeprazole may be used for prevention and treatment of gastrointestinal inflammatory diseases including gastritis, gastric ulcer, and duodenal ulcer. Furthermore, omeprazole may be used for prevention and treatment of other gastrointestinal disorders where cytoprotective and/or gastric antisecretory effect is desirable, e.g. in patients with gastrinomas, acute upper gastrointestinal bleeding, and patients with a history of chronic and excessive alcohol consumption. Omeprazole is also known from U.S. Pat. Nos. 4,738,974; 4,786,505; 4,853,230; 5,690,960; 5,690,960; 5,714,504; 5,714,504; 5,877,192; 5,900,424; 6,147,103; 6,150,380; 6,166,213; 6,191,148; 6,369,085; 6,369,085; and 6,428,810, among others. Lansoprazole is known from U.S. Pat. Nos. 4,628,098; 4,689,333; 5,013,743; 5,026,560; 5,045,321; 5,093,132; 5,433,959; 5,464,632; 6,123,962; and 6,328,994, among others. Rabeprazole is known from U.S. Pat. Nos. 5,035,899 and 5,045,552. Pantoprazole is known from U.S. Pat. Nos. 4,758,579 and 5,997,903, among others. Esomeprazole is known from U.S. Pat. No. 6,428,810 in addition to Omeprazole patents listed above and among others.

U.S. published patent application US20040052847 concerns methods of making oral formulations of drugs having an extremely low solubility in water by converting crystalline active compounds into an amorphous state during coating or spray coating of core particles.

Structurally PPIs contain a sulfinyl group bridging between substituted benzimidazole and pyridine rings. Once these compounds reach the parietal cells and diffuse into the secretory canaliculi, they become protonated. The protonated compounds rearrange to form sulfenic acid and then a sulfenamide. The latter interacts covalently with sulfhydryl groups at critical sites in the extracellular (luminal) domain of the membrane spanning (H⁺, K⁺)-ATPase. Inhibition occurs when two molecules of the inhibitor are bound per molecule of the enzyme. The specificity of these proton pump inhibitors arises from the selective distribution of the (H⁺, K⁺)-ATPase, the acid-catalyzed rearrangement of the compounds to generate the active inhibitor, and the trapping of the protonated compound and the cationic sulfenamide within the acidic canaliculi and adjacent to the target enzyme.

PPIs are typically administered orally as delayed-release dosage forms. The compounds are stable in alkaline pH but are destroyed by gastric acid. Therefore, if the integrity of the enteric coated micro granules or enteric coated non-spherical beads or enteric coated tablets is destroyed in any way and the patient swallows such enteric-coated dosage forms, the acidic pH in the stomach will break down the active compounds. The delayed release dosage forms, when appropriately taken, release the PPIs after the dosage forms leave the stomach.

A variety of adverse reactions have been ascribed to proton pump inhibitors, such as omeprazole and lansoprazole, although the incidence of adverse reactions is low, and the adverse reactions are generally minor. Due to the profound reduction in gastric acidity, there tends to be an increased secretion of gastrin. Hence, patients who take therapeutic doses of PPIs have modest hypergastrinemia. Prolonged administration of high doses of the drugs can cause hyperplasia of oxyntic mucosal cells.

The most common side effects of proton pump inhibitors, such as omeprazole and lansoprazole, are nausea, diarrhea, and abdominal colic. The drugs can also result in bacterial overgrowth in the gastrointestinal tract and the development of nosocomial pneumonia. Omeprazole however is only stable in basic pH conditions and degrades rapidly in acid pH environment and the rate of degradation of lansoprazole in aqueous solution increases with decreasing pH. The degradation half-life of lansoprazole in aqueous solution at 25° C. is approximately 0.5 hour at pH 5.0 and approximately 18 hours at pH 7.0. For this reason the omeprazole and lansoprazole oral dosages form must be protected, not only from the pharmaceutical formulation ingredients acidic in nature used to make a dosage form but also from the acidic gastric fluid in order to reach the absorption site in the small intestine. Manufacturing processes currently employ lengthy enteric coating process times for providing complete gastric protection of drug loaded granules. Also sodium salt forms of rabeprazole and pantoprazole are formulated to provide better stability of these PPIs in tablet dosage forms. Conversion of these PPIs in to their respective salts require additional lengthy manufacturing processing step.

The percent bioavailability of omeprazole from commercially marketed omeprazole dosage forms is 30-40. Lansoprazole, Rabeprazole and Pantoprazole dosage forms provide 80-85%, 52% and 77% respectively of active drugs. Increased bioavailability from the dosage forms help to decrease the daily dose requirements.

Hence, there is a need in the art for proton pump inhibitors that have improved stability of dosage forms, ease in manufacturing techniques, enhanced oral absorption and better gastroprotective properties, decreased the recurrence of ulcers, facilitate ulcer healing and that can be used at low dosages. The present invention is directed to these, as well as other, important ends.

### SUMMARY OF THE INVENTION

The invention provides a composition comprising in admixture:

water,

a pharmaceutically acceptable, volatilizable, organic solvent which is miscible with water;

a non-salt benzimidazole proton pump inhibitor which is soluble in the admixture;

a pharmaceutically acceptable, alkalizing agent; and

a pharmaceutically acceptable, water-soluble, hydrophilic polymer having a surfactant functionality.

The invention also provides a pharmaceutically acceptable particle comprising powder particles comprised of a pharmaceutically acceptable material, said powder particles having coated thereon a composition comprising an admixture of an amorphous, salt form of a benzimidazole proton pump inhibitor produced in-situ; and a pharmaceutically acceptable, water-soluble, hydrophilic polymer having a surfactant functionality.

The composition further provides an oral dosage form comprising:

a core tablet of compressed particles, said compressed particles comprising:

powder particles comprised of a pharmaceutically acceptable material, said

powder particles having coated thereon a composition comprising an admixture of

an amorphous, salt form of a benzimidazole proton pump inhibitor produced in-situ; and
a pharmaceutically acceptable, water-soluble, hydrophilic polymer having a surfactant functionality;
   a pharmaceutically acceptable sub-coating on the core tablet; and
   a pharmaceutically acceptable enteric coating on the sub-coating.

The composition yet further provides a method of producing pharmaceutically acceptable oral dosage form comprising:

(a) forming an admixture comprising:

water,
a pharmaceutically acceptable, volatilizable, organic solvent which is miscible with water;
a non-salt benzimidazole proton pump inhibitor which is soluble in the admixture;
a pharmaceutically acceptable, alkalizing agent;
a pharmaceutically acceptable, water-soluble, hydrophilic polymer having a surfactant functionality;
   (b) coating the composition from (a) onto powder particles comprised of a pharmaceutically acceptable material; combining said coated powder particles with a pharmaceutically acceptable disintegrating agent and a pharmaceutically acceptable lubricant;
   (c) compressing the result from (b) into a core tablet;
   (d) coating said core tablet with a pharmaceutically acceptable sub-coating composition;
   (e) applying a pharmaceutically acceptable enteric coating on the sub-coating.

### DETAILED DESCRIPTION OF THE INVENTION

One aspect of the invention concerns an admixture of water; a pharmaceutically acceptable, volatilizable, organic solvent which is miscible with water; a non-salt benzimidazole proton pump inhibitor which is soluble in the admixture; a pharmaceutically acceptable, alkalizing agent; and a pharmaceutically acceptable, water-soluble, hydrophilic polymer having a surfactant functionality.

As used herein, the term “proton pump inhibitor” refers to any compound that reversibly or irreversibly blocks gastric acid secretion by inhibiting the H⁺/K⁺-ATP ase enzyme system at the secretory surface of the gastric parietal cell.

Useful proton pump inhibitors for use in the present invention non-exclusively include non-salt benzimidazoles, for example, substituted benzimidazoles and substituted azabenzimidazoles, including, for example, omeprazole, lansoprazole, pantoprazole, rabeprazole, leminoprazole, timoprazole, tenatoprazole, disulprazole, esomeprazole and combinations thereof.

In one embodiment of the coating solution composition, the non-salt benzimidazole proton pump inhibitors may be present in the overall solution composition in an amount of from about 0.1% w/v to about 20.0% w/w. In another embodiment, the non-salt benzimidazole proton pump inhibitors may be present in the overall solution composition in an amount of from about 1.0% w/v to about 10.0% w/w. In yet another embodiment, the non-salt benzimidazole—proton pump inhibitors may be present in the overall composition in an amount of from about 2.5% w/w to about 5.0% w/w.

The coating solution composition of the present invention includes at least one pharmaceutically acceptable, alkalizing agent. Alkalizing agents serve to provide an alkaline environment for stabilizing the proton pump inhibitor both for processing and storage and also is used to convert non-salt benzimidazole proton pump inhibitors in to a corresponding salt in the process. Useful alkalizing agent non-exclusively include sodium hydroxide, potassium hydroxide, ammonium hydroxide, disodium hydrogen phosphate, heavy magnesium carbonate, magnesium carbonate, magnesium oxide, magnesium hydroxide, magnesium metasilicate aluminate, magnesium silicate, magnesium aluminate, synthetic hydrotalcite, aluminum magnesium hydroxide, precipitated calcium carbonate, calcium hydroxide, and combinations thereof. In one embodiment, the alkalizing agent component may be present in the overall solution composition in an amount of from about 0.1 moles to about 10 moles per every mole of active. In another embodiment, the alkalizing agent component may be present in the overall solution composition in an amount of from about 0.5 moles to about 5.0 moles per every mole of active. In yet another embodiment, the alkalizing agent component may be present in the overall solution composition in an amount of from about 0.9 moles to about 1.1 moles per mole of non-salt benzimidazole proton pump inhibitor. In one preferred embodiment, the pharmaceutically acceptable, alkalizing agent is present in an amount of from about 1.0 to about 1.05 moles per mole of non-salt benzimidazole proton pump inhibitor.

The overall solution composition further comprises water. In one embodiment, water may be present in the composition in an amount of from 0.5% w/w to about 90.0% w/w. In another embodiment water may be present in the over all solution composition in an amount of from 1.0% w/w to about 20.0% w/w. In still another embodiment water may be present in the solution composition in an amount of from 2.0% w/w to about 6.0% w/w.

The solution composition further comprises a pharmaceutically acceptable, volatilizable, organic solvent which is miscible with water. Useful solvents include alcohols such as methyl alcohol, ethyl alcohol, butyl alcohol, isopropyl alcohol; ketones such as acetone; polyhydric alcohols, glycerin, hexylene glycol, propylene glycol, polyethylene glycol, and combinations thereof. Any suitable acetone may be used to carry out the present invention, such as Pharmacopeial or USP grade acetone. Ethyl alcohol is a preferred solvent. Denatured ethyl alcohol could be used in place of pure ethyl alcohol. In one embodiment, the solvent may be present in the composition in an amount of from about 1.0% w/w to about 90.0% w/w. In another embodiment, the cosolvent may be present in the solution composition in an amount of from about 10.0% w/w to about 88.0% w/w. In still another embodiment the solvent may be present in the solution composition in an amount of from about 80.0% w/w to about 86.0% w/w.

The solution composition further comprises a pharmaceutically acceptable, water-soluble, hydrophilic polymer having a surfactant functionality. Examples of suitable water soluble polymers include, but are not limited to, alkylcelluloses such as methylcellulose, hydroxyalkylcelluloses such as hydroxymethylcellulose, hydroxyethylcellulose, hydroxypropylcellulose and hydroxybutylcellulose; hydroxyalkyl alkylcelluloses such as hydroxyethyl methylcellulose and hydroxypropyl methylcellulose; carboxyalkylcelluloses such as carboxymethylcellulose; alkali metal salts of carboxyalkylcelluloses such as sodium carboxymethylcellulose; carboxyalkylalkylcelluloses such as carboxymethylethylcellulose; carboxyalkylcellulose esters; starches; pectins such as sodium carboxymethylamylopectin; chitin derivatives such as chitosan; polysaccharides such as alginic acid, alkali metal and ammonium salts thereof, carrageenans, galactomannans, traganth, agar-agar, gum arabicum, guar gum and xanthan gum; polyacrylic acids and salts thereof, polymethacrylic acids and salts thereof, including methacrylate copolymers polyvinylpyrrolidone, copolymers of polyvinylpyrrolidone with vinyl acetate; polyalkylene oxides such as polyethylene oxide and polypropylene oxide and copolymers of ethylene oxide and propylene oxide; dextrins and maltodextrins etc. Useful hydroxy propyl methyl cellulose, is manufactured by Aqualon, USA, Dow Chemical Industries, USA and also by Shin-Etsu Chemical Company, Japan, in 5 mPa·s or 3 mPa·s viscosity grades. In one embodiment, the hydrophilic polymer component may be present in the overall solution composition in an amount of from about 1.0% (w/w) to about 20.0% (w/w). In another embodiment, the hydrophilic polymer component may be present in the overall composition in an amount of from about 2.0% (w/w) to about 10.0% w/w. In yet another embodiment, the hydrophilic polymer component may be present in the overall composition in an amount of from about 2.5% (w/w) to about 7.5% w/w.

In the present invention, the water insoluble or very slightly water soluble non-salt benzimidazole is generally converted into an amorphous salt form through the process of dissolving in the solvent and water mixture containing alkalizing agent and hydrophilic polymer and spray coating the solution on pharmaceutically acceptable material in powder form. The top spray granulation process removes the water and solvent mostly and converts the powder particles in to compressible granules. The resultant granules are combined with tablet disintegrating agents and lubricants and then compressed into a core tablet. Core powder particles used herein may be of any suitable size, typically from about 20 to 1000 micrometers in diameter. Examples include particles with a diameter of about 250 to 600 micrometers (60-30 mesh), or a diameter of 600 to 700 micrometers (30-25 mesh). Preferred core powder particles have a diameter of from about 20 micrometers to about 200 micrometers. Size of particles can be determined in accordance with known techniques, such as described in the CRC Handbook, 64th edition, page F-114 and USP24/NF19, page 1969.

The core powder particles may be formed of any suitable pharmaceutically acceptable material. Examples of such materials are polymers e.g., plastic resins; inorganic substances, e.g., silica, glass, hydroxyapatite, salts (sodium or potassium chloride, calcium or magnesium carbonate) and the like; organic substances, e.g., activated carbon, acids (citric, fumaric, tartaric, ascorbic and the like acids), and saccharides and derivatives thereof. Particularly suitable materials are saccharides such as sugars, oligosaccharides, polysaccharides and their derivatives, for example, glucose, rhamnose, galactose, lactose, sucrose, mannitol, sorbitol, dextrin, maltodextrin, cellulose, microcrystalline cellulose, sodium carboxymethyl cellulose, starches (maize, rice, potato, wheat, tapioca) and the like saccharides.

Preferred as a core material for carrying out the present invention is microcrystalline cellulose particles or spheres, which can be produced in accordance with known techniques as described in U.S. Pat. Nos. 4,159,345; 4,149,346; 4,160,014; 4,196,219; 4,199,368; 4,231,802; 4,234,316; 4,275,196; 4,290,911; 4,319,975; 4,330,338; 4,381,082; 4,387,164; 4,415,428; 4,462,839; 4,484,141; 4,504,641; 4,518,433; 4,542,200; 4,588,555; 4,659,672; 4,689,302; 4,693,896; 4,695,548; 4,701,754; 4,717,667; 4,744,987; 4,749,620; 4,774,093; 4,861,448; 4,966,713; 4,983,268; 4,990,611; 5,051,261; 5,053,332; 5,075,115; 5,143,646; 5,155,144; 5,206,030; 5,212,299; 5,258,436; 5,277,915; 5,326,572; etc.

Useful microcrystalline cellulose powder particles in the form of AVICEL™ is available from FMC Corporation.

Tablets can be produced by conventional tabletting techniques with conventional ingredients or excipients. The tablets are preferably formed from a composition comprising the particles described herein distributed in a mixture of a disintegrating agent and a diluent or filler. Suitable diluents include, but are not limited to, lactose, sucrose, dextrose, mannitol, sorbitol, starch, cellulose, calcium phosphate, microcrystalline cellulose such as AVICEL™ etc. Tablets may include a variety of other conventional ingredients, such as binders, buffering agents, lubricants, glidants, thickening agents, sweetening agents, plasticizers, flavors, pigments, preservatives, complexing and chelating agents, electrolytes or other active ingredients in amounts of up to about 10 percent by weight based on the weight of the compressed tablet.

Useful lubricants non-exclusively include magnesium stearate, talc, stearic acid, polyethylene glycol, glyceryl behenate, zinc stearate, and vegetable oil derivatives and may be present in an amount of from about 0.1% by weight to about 5.0 percent by weight based on the weight of the compressed tablet.

Useful disintegrating agents non-exclusively include but are not limited to, crospovidone, croscarmellose sodium, sodium starch glycolate, various grades of starch, polacrilin potassium and may be present in an amount of from about 0.2% by weight to about 10.0 percent by weight based on the weight of the compressed tablet.

The compressed tablet is then preferably provided with a sealing sub-coating to separate the compressed tablet from a subsequently applied enteric coating. The sealing sub-coating protects the tablet active ingredients from chemical interactions with the enteric coating dispersion ingredients and thereby rendering proton pump inhibitors not to undergo acid catalyzed chemical degradation.

Useful compositions for this purpose are well known in the art and are generally commercially available. The protective sub-coating can be applied by a standard film coating procedure in a suitable coating machine using aqueous dispersions containing hydroxypropyl methylcellulose, polyethylene glycol, hydroxyethyl cellulose, hydroxypropyl cellusose, and polyvinylpyrrolidone. One useful sub-coating is OPADRY® which is commercially available from Colorcon of West Point, Pa. An alkaline environment is provided in the sub-coat by incorporation of disodium hydrogen phosphate in the solution prior to sub-coating.

The compressed, sub-coated tablet is then applied with an enteric coating to deter disintegration in the stomach and the enteric polymer starts dissolving as the dosage units leave the stomach of the subject. The enteric coat surrounds the core dosage form with a film which is hydrophobic at acidic pH values. Enteric coatings are well known in the art. Such materials can include polymers, plasticizers, and optional excipients.

Suitable polymers for the enteric coating of this invention are insoluble in acidic environments (e.g., gastric juice) but are soluble at pH 5.5 and upwards. Such polymers include cellulose acetate phthalate, methacrylate-base polymers, cellulose acetate trimellitate, hydroxypropyl methylcellulose acetate succinate, hydroxypropyl methylcellulose phthalate, polyvinyl acetate phthalate, anionic phthalate polymers based on methacrylic acid and methacrylic acid esters, and the like. These compounds are either used alone or in combination in an organic solvent. Generally, the polymers are dissolved in organic solvents before being used in a film coating process. For example, they include methyl alcohol, ethyl alcohol, ethyl alcohol/water, isopropyl alcohol, isopropyl alcohol/water, n-butyl alcohol, propylene glycol, ethyleneglycol monobutyl ether, acetone, acetone/isopropyl alcohol, and the like. Aqueous based polymeric dispersions are preferred for enteric coating applications in pharmaceutical industries. Suitable plasticizers impart sufficient tensile strength to the coating to prevent film cracking. Such plasticizers include triethyl citrate, dibutyl phthalate, polyethylene glycols, propylene glycol, diethylphthalate, acetyl triethyl citrate, and the like. The coating procedures are performed in a suitable coating machine. Omeprazole and lansoprazole delayed release dosage forms (in capsules) are official in United States Pharmacopoeia (USP 28-NF23). Assay and drug release tests for the invention tablets and marketed capsule formulation are performed following the USP procedures. Purity and related substances test is also conducted to evaluate the quality of the invention dosage forms.

Useful enteric coatings are also commercially available. Such include SURETERIC® and ACRYL-EZE™, both of which are commercially available from Colorcon of West Point, Pa.

Drug release of the delayed release (enteric coated) tablets (invention) data in comparison to that of marketed capsule formulation indicate that the invention tablets have consistently higher dissolution results and also accelerated stability of the invention tablets prove that the composition and process used in the manufacturing of invention tablets produce superior quality drug product as compared to marketed formulation. Better dissolution may provide better absorption from the invention tablets of the actives.

Subjects afflicted with a disorder that may be treated with the oral dosage forms described herein include both human subjects and mammalian subjects such as dogs, cats and rabbits, etc. Disorders with which such subjects may be afflicted include those for which the proton pump inhibitor compounds described above are known to be effective in treating. The dosage of proton pump inhibitor compounds will vary depending on factors such as the disease and severity thereof, the age, weight and condition of the subject, etc., but in some embodiments is from about 5.0 milligrams per unit dosage form to about 80.0 milligrams per unit dosage form. The dosage form or forms may be administered to the subject at a single time or (more preferably) on multiple occasions over the day, and may be administered to the subjects under fed conditions, that is, simultaneously with food, or shortly before or after the subject has eaten so that the residence time of the dosage form in the subject's stomach is longer as compared to fasted conditions, or may be administered to the subject under fasted conditions, that is, without concurrent food administration so that the residence time of the dosage form in the subject's stomach is shorter as compared to fed conditions.

The following non-limiting examples serve to illustrate the invention.

### EXAMPLE 1

### Omeprazole Delayed Release Tablets 20 mg

This example describes the preparation of an oral delayed release tablet dosage form of omeprazole in accordance with the methods of the present invention.

Ingredients: Ingredients for the preparation of omeprazole delayed release Tablets 20 mg of the invention are set forth in the table below.

Core Tablet:

| | | Formula weight - |
|---|---|---|
| Per Unit | | 1000 core |
| in mg | Ingredient | tablets in g |
| 37.2 | Hydroxypropyl Methyl Cellulose (¹) | 37.2 |
| 20.0 | Omeprazole | 20.0 |
| 0.0 | Ethyl Alcohol (²) | 514.5 |
| 0.0 | Purified Water (²) | 27.0 |
| 2.4 | Sodium Hydroxide | 2.4 |
| 301.7 | Microcrystalline Cellulose Powder (³) | 301.7 |
| 15.2 | Croscarmellose Sodium | 15.2 |
| 2.0 | Magnesium Stearate | 2.0 |
| 1.5 | Talc | 1.5 |
| 380.0 | Core Tablet Total Weight | 380.0 |

(¹) Hydroxy propyl methyl cellulose of 3 centipoises viscosity grade was used
(²) Removed during processing and ethyl alcohol could be of denatured grade
(³) Avicel ® pH102 grade was used

Process: Ethyl alcohol 514.5 g was taken in to a stainless steel container. Hydroxypropyl methyl cellulose was suspended in alcohol under stirring. In a separate container purified water 27.0 g was taken and sodium hydroxide 2.4 g was dissolved. Sodium hydroxide solution was added to alcohol medium under stirring. The drug omeprazole 20.0 g was added in to the above mixture under stirring. The stirring was continued until the entire drug was dissolved.

A STREA-1 fluid bed processor equipped with a top spray insert was used for spray coating of the liquid onto microcrystalline cellulose powder 301.7 g. A spray rate of 2.0 to 2.5 g per minute was used. Drug loading was performed at a product bed temperature of 28-35° C. with an air volume of 60-70 cubic meters per hour and atomizing air pressure of 2.2 to 2.6 bar. The drug loaded particles were dried for an additional 15 minutes at a product bed temperature not exceeding 35° C. to obtain a proper loss on drying value of between 1.0 to 3.0 percent.

Drug loaded particles were combined with croscarmellose sodium 15.2 g, magnesium stearate 2.0 g and talc 1.5 g by blending in an appropriate blender. The blended granules were compressed in to a core tablet with an average weight of 380.0 mg using a tablet press with 11.2 mm diameter shallow concave punch tooling at an average hardness of 10 kg/cm² with a thickness range of 4.5 to 4.7 mm.

Sub-coating: Ingredients

| | | Formula weight - |
|---|---|---|
| Per Unit | | 1000 sub-coated |
| in mg | Ingredient | tablets in g |
| 380.0 | Core Tablet | 380.0 |
| 5.517 | Opadry-03K19299 (¹) | 5.517 |
| 0.184 | Disodium Hydrogen Phosphate | 0.184 |
| 0.0 | Purified Water (²) | 67.85 |
| 385.7 | Sub-Coated Tablet Total Weight | 385.7 |

(¹) Opadry ® is supplied by Colorcon
(²) Removed during the process

Process: The sub-coating material Opadry® at 7.5% was dispersed in purified water under constant stirring. Disodium hydrogen phosphate was added and dissolved. The dispersion was sprayed using a coating pan (O'Hara 15″) with baffles and at an atomization air pressure of 20 PSI; at an air flow of 165 CFM, product bed temperature of 42-51° C. and until the tablets obtain a weight gain of 1.5%.

Delayed Release (Enteric) Coating: Ingredients

| | | Formula weight - |
|---|---|---|
| Per Unit | | 1000 enteric coated |
| in mg | Ingredient | tablets in g |
| 385.7 | Sub-coated Tablet | 385.7 |
| 30.9 | AcrylEze-93F19255 (¹) | 30.9 |
| 0.0 | Purified Water (²) | 123.6 |
| 416.6 | Final Enteric Coated Tablet | 416.6 |

(¹) AcrylEze ® is supplied by Colorcon
(²) Removed during the process

Process: 20.0 percent by weight suspension in water of Acryl-Eze (commercially available from Colorcon of West Point Pennsylvania) was sprayed using a coating pan (O'Hara 15″) with baffles and at an atomization air pressure of 20 PSI; at an air flow of 165 CFM, product bed temperature of 42-51° C. and until the tablets obtain a weight gain of 8.0%.

### EXAMPLE 2

### Lansoprazole Delayed Release Tablets 30 mg

This example describes the preparation of an oral delayed release tablet dosage form of Lansoprazole in accordance with the methods of the present invention.

Ingredients: Ingredients for the preparation of Lansoprazole Delayed Release Tablets 30 mg of the invention are set forth in the table below.

Core Tablet:

| | | Formula weight - |
|---|---|---|
| Per Unit | | 1000 core |
| in mg | Ingredient | tablets in g |
| 54.8 | Hydroxypropyl Methyl Cellulose (¹) | 54.8 |
| 30.0 | Lansoprazole | 30.0 |
| 0.0 | Ethyl Alcohol (²) | 756.0 |
| 0.0 | Purified Water (²) | 40.0 |
| 3.36 | Sodium Hydroxide | 3.36 |
| 456.04 | Microcrystalline Cellulose Powder (³) | 456.04 |
| 30.0 | Croscarmellose Sodium | 30.0 |
| 3.4 | Magnesium Stearate | 3.4 |
| 2.4 | Talc | 2.4 |
| 580.0 | Core Tablet Total Weight | 580.0 |

(¹) Hydroxy propyl methyl cellulose of 3 centipoises viscosity grade was used
(²) Removed during processing and ethyl alcohol could be of denatured grade
(³) Avicel ® pH102 grade was used

Process: Ethyl alcohol 756.0 g was taken in to a stainless steel container. hydroxypropyl methyl cellulose was suspended in alcohol under stirring. In a separate container purified water 40.0 g was taken and sodium hydroxide 3.36 g was dissolved. Sodium hydroxide solution was added to alcohol medium under stirring. The drug lansoprazole 30.0 g was added in to the above mixture under stirring. The stirring was continued until the entire drug was dissolved.

A STREA-1 fluid bed processor equipped with a top spray insert was used for spray coating of the liquid on to microcrystalline cellulose powder 456.04 g. Spray rate of 2.0 to 2.5 g per minute was used. Drug loading was performed at a product bed temperature of 28-35° C. with an air volume of 60-70 Cubic Meters per Hour and atomizing air pressure of 2.2 to 2.6 bar. The drug loaded particles were dried for an additional 15 minutes at a product bed temperature not exceeding 35° C. to obtain a proper loss on drying value of between 1.0 to 3.0 percent.

Drug loaded particles were combined with croscarmellose sodium 30.0 g magnesium stearate 3.4 g and Talc 2.4 g by blending in an appropriate blender. The blended granules were compressed in to a core tablet with an average weight of 580.0 mg using a tablet press with 11.2 mm diameter shallow concave punch tooling at average hardness of 10 kg/cm² with a thickness range of 6.0 to 6.5 mm.

Sub-coating: Ingredients

| | | Formula weight - |
|---|---|---|
| Per Unit | | 1000 sub-coated |
| in mg | Ingredient | tablets in g |
| 580.0 | Core Tablet | 580.0 |
| 8.42 | Opadry-03K19299 (¹) | 8.42 |
| 0.28 | Disodium Hydrogen Phosphate | 0.28 |
| 0.0 | Purified Water (²) | 103.5 |
| 588.7 | Sub-Coated Tablet Total Weight | 588.7 |

(¹) Opadry ® is supplied by Colorcon
(²) Removed during the process

Process: The sub-coating material Opadry® at 7.5% was dispersed in purified water under constant stirring. Disodium hydrogen phosphate was added and dissolved. The dispersion was sprayed using a coating pan (O'Hara 15″) with baffles and at an atomization air pressure of 20 PSI; at an air flow of 165 CFM, product bed temperature of 42-51° C. and until the tablets obtain a weight gain of 1.5%.

Delayed Release (Enteric) Coating Ingredients:

| | | Formula weight - |
|---|---|---|
| Per Unit | | 1000 enteric coated |
| in mg | Ingredient | tablets in g |
| 588.7 | Sub-coated Tablet | 588.7 |
| 47.0 | AcrylEze-93F19255 (¹) | 47.0 |
| 0.0 | Purified Water (²) | 188.0 |
| 635.7 | Final Enteric Coated Tablet | 635.7 |

(¹) AcrylEze ® is supplied by Colorcon
(²) Removed during the process

Process: 20.0 percent by weight suspension in water of Acryl-Eze® (commercially available from Colorcon of West Point Pennsylvania) was sprayed using a coating pan (O'Hara 15″) with baffles and at an atomization air pressure of 20 PSI; at an air flow of 165 CFM, product bed temperature of 42-51° C. and until the tablets obtain a weight gain of 8.0%.

### EXAMPLE 3

### Omeprazole Delayed Release Tablets 10 mg

This example describes the preparation of an oral delayed release tablet dosage form of Omeprazole in accordance with the methods of the present invention.

Ingredients: Ingredients for the preparation of Omeprazole delayed release Tablets 10 mg of the invention are set forth in the table below.

Core Tablet:

| | | Formula weight - |
|---|---|---|
| Per Unit | | 2000 core tablets |
| in mg | Ingredient | in g |
| 18.6 | Hydroxypropyl Methyl Cellulose (¹) | 37.2 |
| 10.0 | Omeprazole | 20.0 |
| 0.0 | Ethyl Alcohol (²) | 514.5 |
| 0.0 | Purified Water (²) | 27.0 |
| 1.2 | Sodium Hydroxide | 2.4 |
| 150.85 | Microcrystalline Cellulose Powder (³) | 301.7 |
| 7.6 | Croscarmellose Sodium | 15.2 |
| 1.0 | Magnesium Stearate | 2.0 |
| 0.75 | Talc | 1.5 |
| 190 | Core Tablet Total Weight | 380.0 |

(¹) Hydroxy Propyl Methyl Cellulose of 3 centipoises viscosity grade was used
(²) Removed during processing and Ethyl Alcohol could be of denatured grade
(³) Avicel ® pH102 grade was used

For process, refer example 1 and compress the tablets using 9.5 mm punch tooling.

Sub-coating: Ingredients:

| | | Formula weight - |
|---|---|---|
| Per Unit | | 2000 sub-coated |
| in mg | Ingredient | tablets in g |
| 190.0 | Core Tablet | 380.0 |
| 2.76 | Opadry-03K19299 (¹) | 5.517 |
| 0.092 | Disodium Hydrogen Phosphate | 0.184 |
| 0.0 | Purified Water (²) | 67.85 |
| 192.85 | Sub-Coated Tablet Total Weight | 385.7 |

(¹) Opadry ® is supplied by Colorcon
(²) Removed during processing

For sub-coating process, refer example 1.

Delayed Release (Enteric) Coating Ingredients:

| | | Formula weight - |
|---|---|---|
| Per Unit | | 2000 enteric coated |
| in mg | Ingredient | tablets in g |
| 192.85 | Sub-coated Tablet | 385.7 |
| 15.45 | AcrylEze-93F19255 (¹) | 30.9 |
| 0.0 | Purified Water (²) | 123.6 |
| 208.3 | Final Enteric Coated Tablet | 416.6 |

(¹) AcrylEze ® is supplied by Colorcon
(²) Removed during processing
For enteric coating process, refer to Example 1

### EXAMPLE 4

### Omeprazole Delayed Release Tablets 40 mg

This example describes the preparation of an oral delayed release tablet dosage form of Omeprazole in accordance with the methods of the present invention.

Ingredients: Ingredients for the preparation of Omeprazole Delayed Release Tablets 40 mg of the invention are set forth in the table below.

Core Tablet:

| | | Formula weight - |
|---|---|---|
| Per Unit | | 500 core tablets |
| in mg | Ingredient | in g |
| 74.4 | Hydroxypropyl Methyl Cellulose (¹) | 37.2 |
| 40.0 | Omeprazole | 20.0 |
| 0.0 | Ethyl Alcohol (²) | 514.5 |
| 0.0 | Purified Water (²) | 27.0 |
| 4.8 | Sodium Hydroxide | 2.4 |
| 603.4 | Microcrystalline Cellulose Powder (³) | 301.7 |
| 30.4 | Croscarmellose Sodium | 15.2 |
| 4.00 | Magnesium Stearate | 2.0 |
| 3.00 | Talc | 1.5 |
| 760.0 | Core Tablet Total Weight | 380.0 |

(¹) Hydroxy Propyl Methyl Cellulose of 3 centipoises viscosity grade was used
(²) Removed during processing and Ethyl Alcohol could be of denatured grade
(³) Avicel ® pH102 grade was used

For the process, refer to Example 1 and compress using a 12.5 mm punch tooling.

Sub-coating: Ingredients

| | | Formula weight - |
|---|---|---|
| Per Unit | | 500 sub-coated |
| in mg | Ingredient | tablets in g |
| 760.0 | Core Tablet | 380.0 |
| 11.034 | Opadry-03K19299 (¹) | 5.517 |
| 0.368 | Disodium Hydrogen Phosphate | 0.184 |
| 0.0 | Purified Water (²) | 67.85 |
| 771.40 | Sub-Coated Tablet Total Weight | 385.7 |

(¹) Opadry ® is supplied by Colorcon
(²) Removed during processing

For sub-coating process, refer example 1.

Delayed Release (Enteric) Coating: Ingredients:

| | | Formula weight - |
|---|---|---|
| Per Unit | | 500 enteric coated |
| in mg | Ingredient | tablets in g |
| 771.40 | Sub-coated Tablet | 385.7 |
| 61.8 | AcrylEze-93F19255 (¹) | 30.9 |
| 0.0 | Purified Water (²) | 123.6 |
| 833.2 | Final Enteric Coated Tablet | 416.6 |

(¹) AcrylEze ® is supplied by Colorcon
(²) Removed during processing
For enteric coating process, refer example 1.

### EXAMPLE 5

### Lansoprazole Delayed Release Tablets 15 mg

This example describes the preparation of an oral delayed release tablet dosage form of Lansoprazole in accordance with the methods of the present invention.

Ingredients: Ingredients for the preparation of Lansoprazole Delayed Release Tablets 15 g of the invention are set forth in the table below.

Core Tablet:

| | | Formula weight - |
|---|---|---|
| Per Unit | | 1000 core |
| in mg | Ingredient | tablets in g |
| 27.4 | Hydroxypropyl Methyl Cellulose (¹) | 54.8 |
| 15.0 | Lansoprazole | 30.0 |
| 0.0 | Ethyl Alcohol (²) | 756.0 |
| 0.0 | Purified Water (²) | 40.0 |
| 1.68 | Sodium Hydroxide | 3.36 |
| 228.02 | Microcrystalline Cellulose Powder (³) | 456.04 |
| 15 | Crosscarmellose Sodium | 30.0 |
| 1.7 | Magnesium Stearate | 3.4 |
| 1.2 | Talc | 2.4 |
| 290.0 | Core Tablet Total Weight | 580.0 |

(¹) Hydroxy Propyl Methyl Cellulose of 3 centipoises viscosity grade was used
(²) Removed during processing and Ethyl Alcohol could be of denatured grade
(³) Avicel ® pH102 grade was used

For the process, refer to Example 2 and use a 9.5 mm punch tooling to compress the tablets.

Sub-coating: Ingredients

| | | Formula weight - |
|---|---|---|
| Per Unit | | 1000 sub-coated |
| in mg | Ingredient | tablets in g |
| 290.0 | Core Tablet | 580.0 |
| 4.21 | Opadry-03K19299 (¹) | 8.42 |
| 0.14 | Disodium Hydrogen Phosphate | 0.28 |
| 0.0 | Purified Water (²) | 103.5 |
| 294.35 | Sub-Coated Tablet Total Weight | 588.7 |

(¹) Opadry ® is supplied by Colorcon
(²) Purified Water is removed in the process

For the sub-coating process, refer to Example 2.

Delayed Release (Enteric) Coating: Ingredients

| | | Formula weight - |
|---|---|---|
| Per Unit | | 1000 enteric coated |
| in mg | Ingredient | tablets in g |
| 294.35 | Sub-coated Tablet | 588.7 |
| 23.5 | AcrylEze-93F19255 (¹) | 47.0 |
| 0.0 | Purified Water (²) | 188.0 |
| 317.85 | Final Enteric Coated Tablet | 635.7 |

(¹) AcrylEze ® is supplied by Colorcon
(²) Purified Water was removed in the process

For the enteric coating process, refer to Example 2.

### EXAMPLE 6

### Omeprazole Delayed Release Tablets 20 mg with Ammonium Hydroxide

This example describes the preparation of an oral delayed release tablet dosage form of Omeprazole in accordance with the methods of the present invention.

Ingredients: Ingredients for the preparation of Omeprazole Delayed Release Tablets 20 mg of the invention are set forth in the table below.

Core Tablet:

| | | Formula weight - |
|---|---|---|
| Per Unit | | 1000 core |
| in mg | Ingredient | tablets in g |
| 37.2 | Hydroxypropyl Methyl Cellulose (¹) | 37.2 |
| 0.00 | Ammonium Hydroxide | 3.6 |
| 20.0 | Omeprazole | 20.0 |
| 0.0 | Ethyl Alcohol (²) | 525.9 |
| 0.0 | Purified Water (²) | 10.2 |
| 2.1 | Di Sodium Hydrogen Phosphate | 2.1 |
| 317.2 | Microcrystalline Cellulose Powder (³) | 317.2 |
| 2.0 | Magnesium Stearate | 2.0 |
| 1.5 | Talc | 1.5 |
| 380.0 | Core Tablet Total Weight | 380.0 |

(¹) Hydroxy Propyl Methyl Cellulose of 3 centipoises viscosity grade was used
(²) Removed during processing and Ethyl Alcohol could be of denatured grade
(³) Avicel ® pH102 grade was used

For top spray granulation and compression, sub-coating and delayed release ingredients and processes, refer to Example 1.

### EXAMPLE 7

### Lansoprazole Delayed Release Tablets 30 mg with Ammonium Hydroxide

This example describes the preparation of an oral delayed release tablet dosage form of Lansoprazole in accordance with the methods of the present invention.

Ingredients: Ingredients for the preparation of Lansoprazole Delayed Release Tablets 30 mg of the invention are set forth in the table below.

Core Tablet:

| | | Formula weight - |
|---|---|---|
| Per Unit | | 1000 core tablets |
| in mg | Ingredient | in g |
| 54.8 | Hydroxypropyl Methyl Cellulose (¹) | 54.80 |
| 0.0 | Ammonium Hydroxide | 5.30 |
| 30 | Lansoprazole | 30.00 |
| 0.0 | Ethyl Alcohol (²) | 777.47 |
| 0.0 | Purified Water (²) | 12.00 |
| 3.10 | Di Sodium Hydrogen Phosphate | 3.10 |
| 456.3 | Microcrystalline Cellulose Powder (³) | 456.3 |
| 30 | Croscarmellose Sodium | 30.00 |
| 3.4 | Magnesium Stearate | 3.40 |
| 2.4 | Talc | 2.40 |
| 580.0 | Core Tablet Total Weight | 580.00 |

(¹) Hydroxy Propyl Methyl Cellulose of 3 centipoises viscosity grade was used
(²) Removed during processing and Ethyl Alcohol could be of denatured grade
(³) Avicel ® pH102 grade was used

For top spray granulation and compression, sub-coating and delayed release ingredients and processes, refer to Example 2.

### EXAMPLE 8

Tablets of the invention prepared according to Example 1 were compared with marketed capsule dosage forms for their dissolution in the pH 6.8 phosphate buffer after exposing them for two hours in 0.1 N hydrochloric acid for 2 hours and the testing is performed as per the monograph under Omeprazole delayed release capsules in USP 28 using paddle method.

TABLE -1
Omeprazole Drug Release Comparison:
Buffer Stage
Invention tablets vs. Market capsules
Conditions: USP Type 2 (Paddles, RPM 100, Volume 900 mL)
Medium Used: 0.1N Hydrochloric Acid for 2 hours followed by pH 6.8 Phosphate buffer

| Time | % Quantity of Omeprazole dissolved | | | | | | | | |
|---|---|---|---|---|---|---|---|---|---|
| Interval | Sample | 1 | 2 | 3 | 4 | 5 | 6 | Min. | Max |
| 15 min | Invention Tablets | 85.6 | 87.1 | 86.4 | 83.4 | 88.7 | 89.7 | 83.4 | 89.7 |
| | Marketed Capsules | 56.4 | 65.9 | 66.1 | 63.2 | 61.0 | 59.5 | 56.4 | 65.9 |
| 30 min | Invention Tablets | 95.2 | 94.6 | 96.7 | 94.7 | 93.8 | 96.5 | 93.8 | 96.7 |
| | Marketed Capsules | 85.9 | 86.4 | 86.2 | 83.5 | 80.1 | 87.2 | 80.1 | 87.2 |

TABLE 2
Omeprazole Drug Release Comparison:
Acid Resistance Stage
Invention tablets vs. Market capsules
Conditions: USP Type 2 (Paddles, RPM 100 Volume 500 mL)
Medium Used: 0.1N Hydrochloric Acid
% Drug Release in 0.1N Hydrochloric Acid after 2 hours

| | Invention Tablets | Marketed Capsules |
|---|---|---|
| Unit No. | Per unit | Per unit |
| Unit 1 | 1.1 | 0.8 |
| Unit 2 | 0.4 | 1.3 |
| Unit 3 | 1.3 | 0.7 |
| Unit 4 | 1.5 | 2.4 |
| Unit 5 | 1.4 | 1.1 |
| Unit 6 | 0.9 | 2.6 |

The results in Table 1 clearly indicate that the invention formulation of omeprazole tablets 20 mg dissolves completely in pH 6.8 buffer medium with very good gastric resistance (good protection) in acidic environment as is evident from the Table 2 gastric resistance values.

### EXAMPLE 9

Lansoprazole tablets 30 mg prepared according to Example 2 were tested for dissolution and gastric resistance as per the procedure described under Lansoprazole delayed release capsules in USP 28 and the results are furnished in Tables 3 and 4 respectively. These results clearly indicate that the formulation performs very well in in-vitro dissolution testing of delayed release dosage forms.

TABLE -3
Lansoprazole Drug Release
Buffer Stage
Invention tablets
Conditions: USP Type 2 (Paddles, RPM 75, Volume 900 Ml)
Medium Used: 0.1N Hydrochloric Acid for 1 hour followed by buffer

| Time | % Quantity of Lansoprazole dissolved | | | | | | | |
|---|---|---|---|---|---|---|---|---|
| Interval | 1 | 2 | 3 | 4 | 5 | 6 | Min. | Max. |
| 15 min | 47.2 | 49.5 | 54.7 | 43.9 | 57.2 | 48.1 | 43.9 | 57.2 |
| 30 min | 94.8 | 98.9 | 101.0 | 99.0 | 99.3 | 102.6 | 94.8 | 102.6 |
| 45 min | 97.0 | 100.0 | 102.6 | 99.6 | 101.0 | 102.0 | 97.0 | 102.6 |
| 60 min | 96.4 | 99.9 | 102.5 | 100.2 | 100.9 | 102.2 | 96.4 | 102.5 |
| 90 min | 97.4 | 100.4 | 103.5 | 100.8 | 101.8 | 102.0 | 97.4 | 103.5 |

TABLE 4
Lansoprazole Drug Release
Acid Resistance Stage
Invention tablets
Conditions: USP Type 2 (Paddles, RPM 75 Volume 500 Ml)
Medium Used: 0.1N Hydrochloric Acid
% Drug Release in 0.1N Hydrochloric Acid after 1 hours

| | Invention Tablets |
|---|---|
| Unit No. | Per unit |
| Unit 1 | 1.7 |
| Unit 2 | 2.1 |
| Unit 3 | 1.3 |
| Unit 4 | 0.5 |
| Unit 5 | 0.6 |
| Unit 6 | 0.1 |

### EXAMPLE 10

The enteric tablets obtained according to Examples 1 and 2 and a reference sample were stored at 40° C. and 75% R for two weeks and the appearance of each tablet was observed. Table 5 shows results.

TABLE 5
Accelerated Conditions of 40° C. and 75% RH

| Sample details | First Week | Second Week |
|---|---|---|
| Example 1 tablets | − | − |
| Example 2 tablets | − | − |
| Reference Example | ± | ± |

Note:
− not changed (white)
± some what changed

While the present invention has been particularly shown and described with reference to preferred embodiments, it will be readily appreciated by those of ordinary skill in the art that various changes and modifications may be made without departing from the spirit and scope of the invention. It is intended that the claims be interpreted to cover the disclosed embodiment, those alternatives which have been discussed above and all equivalents thereto.

1-15. (canceled)

16. An oral dosage form comprising:
a core tablet of compressed granules, said compressed granules comprising:
powder particles comprised of a pharmaceutically acceptable material, said powder particles having coated thereon a composition comprising, in admixture:
an amorphous, salt form of a benzimidazole proton pump inhibitor produced in-situ; and
a pharmaceutically acceptable, water-soluble hydrophilic polymer having a surfactant functionality;
a pharmaceutically acceptable sub-coating on the core tablet; and
a pharmaceutically acceptable enteric coating on the sub-coating.

17. The oral dosage form of claim 16 wherein said core tablet of compressed granules further comprises at least one pharmaceutically acceptable disintegrating agent of and/or pharmaceutically acceptable lubricant.

18. The oral dosage form of claim 16 comprising an amorphous salt form of a omeprazole, pantoprazole, rabeprazole, leminoprazole, lansoprazole, timoprazole, tenatoprazole, disulprazole, esomeprazole, and combinations thereof.

19. The oral dosage form of claim 16 comprising an amorphous salt form of omeprazole.

20. The oral dosage form of claim 16 comprising an amorphous salt form of lansoprazole.

21. The oral dosage form of claim 16 wherein the pharmaceutically acceptable water-soluble, hydrophilic polymer having a surfactant functionality comprises hydroxypropyl methyl cellulose.

22. The oral dosage form of claim 16 wherein the powder particles comprise microcrystalline cellulose.

23. The oral dosage form of claim 16 wherein the powder particles comprise microcrystalline cellulose; the proton pump inhibitor comprises an amorphous salt form of omeprazole or lansoprazole; and the pharmaceutically acceptable water-soluble, hydrophilic polymer having a surfactant functionality comprises hydroxypropyl methyl cellulose.

24. A method of treating a disorder in a subject in need thereof, comprising orally administering to said subject an oral dosage form according to claim 16 in a pharmaceutically acceptable amount.

25. A method of producing pharmaceutically acceptable oral dosage form comprising:
(a) forming a solution composition comprising, in admixture:
water,
a pharmaceutically acceptable volatilizable, organic solvent which is miscible with water;
a salt form of a benzimidazole proton pump inhibitor formed by the reaction of a non-salt benzimidazole proton pump inhibitor and a pharmaceutically acceptable, alkalizing agent; and
a pharmaceutically acceptable, water-soluble, hydrophilic polymer having a surfactant functionality;
(b) coating the solution composition from (a) onto powder particles comprised of a pharmaceutically acceptable material, removing water and solvent from the solution composition on the powder particles to thereby form compressible granules; combining said compressible granules with a pharmaceutically acceptable disintegrating agent and/or a pharmaceutically acceptable lubricant;
(c) compressing the result from (b) into a core tablet;
(d) coating said core tablet with a pharmaceutically acceptable sub-coating composition;
(e) applying a pharmaceutically acceptable enteric coating on the sub-coating.

26. The method of claim 25 wherein the salt form of benzimidazole proton pump inhibitor comprises an amorphous salt form of an omeprazole, pantoprazole, rabeprazole, leminoprazole, lansoprazole, timoprazole, tenatoprazole, disulprazole, esomeprazole, and combinations thereof.

27. The method of claim 25 wherein the pharmaceutically acceptable, volatilizable, organic solvent comprises an alcohol, a ketone, or combinations thereof.

28. The method of claim 25 wherein the pharmaceutically acceptable, alkalizing agent is selected from the group consisting of sodium hydroxide, potassium hydroxide, ammonium hydroxide, disodium hydrogen phosphate, heavy magnesium carbonate, magnesium carbonate, magnesium oxide, magnesium hydroxide, magnesium metasilicate aluminate, magnesium silicate, magnesium aluminate, synthetic hydrotalcite, aluminum magnesium hydroxide, precipitated calcium carbonate, calcium hydroxide, and combinations thereof.

29. The method of claim 25 wherein the pharmaceutically acceptable, water-soluble, hydrophilic polymer having a surfactant functionality comprises hydroxypropyl methyl cellulose.

30. The method of claim 25 wherein the salt form of benzimidazole proton pump inhibitor comprises omeprazole or lansoprazole; the pharmaceutically acceptable, volatilizable, organic solvent comprises ethanol, pharmaceutically acceptable, alkalizing agent comprises sodium hydroxide; and the pharmaceutically acceptable water-soluble hydrophilic polymer having a surfactant functionality comprises hydroxypropyl methyl cellulose.

31. The method of claim 25 wherein the pharmaceutically acceptable, alkalizing agent is present in an amount of from about 1.0 to about 1.05 moles per mole of non-salt benzimidazole proton pump inhibitor.

32. The method of claim 25 wherein the powder particles comprise microcrystalline cellulose.

33. The method of claim 25 wherein the powder particles comprise microcrystalline cellulose; the proton pump inhibitor comprises an amorphous salt form of omeprazole or lansoprazole; and the pharmaceutically acceptable, water-soluble, hydrophilic polymer having a surfactant functionality comprises hydroxypropyl methyl cellulose.

34. The method of claim 25 wherein the powder particles have a diameter of from about 20 micrometers to about 200 micrometers.

35. The oral dosage form of claim 16 wherein the powder particles have a diameter of from about 20 micrometers to about 200 micrometers.
